(19) United States
(12) Patent Application Publication
(10) Pub. No.: US 2024/0230101 A1
(43) Pub. Date: Jul. 11, 2024

(54) PARTICLE EXHAUST SENSOR FOR A SOLID FUEL-BURNING APPLIANCE AND SOLID FUEL-BURNING APPLIANCE INCLUDING SAME

(71) Applicant: FABRICANT DE POÊLES INTERNATIONAL INC., Saint-Augustin-de Desmaures (CA)

(72) Inventors: Guillaume Thibodeau-Fortin, Saint-Augustin-de Desmaures (CA); Nicolas Gagnon, Saint-Augustin-de Desmaures (CA); Charles Richard, La Pocatière (CA); Guillaume Caron, Saint-Augustin-de Desmaures (CA); Leo Charest, La Pocatière (CA); Adam Roy, La Pocatière (CA); Simon Fissette, La Pocatière (CA)

(21) Appl. No.: 18/400,932

(22) Filed: Dec. 29, 2023

Related U.S. Application Data
(60) Provisional application No. 63/478,570, filed on Jan. 5, 2023.

(51) Int. Cl. F24B 5/02; F23N 3/00; G01K 3/00; G01N 15/075; G01N 33/00

(52) U.S. Cl. CPC F24B 5/021; F23N 3/002; G01K 3/005; G01N 15/075; G01N 33/0027

(57) ABSTRACT

Exhaust sensors for use with residential solid fuel-burning appliances are disclosed. In one example, a sensor apparatus includes an optical sensor, a lens, and first and second light sources positioned adjacent to the optical sensor. The first light source is configured to emit a first light signal towards a target area of an exhaust gas stream. The second light source is configured to emit a second light signal towards the target area. The optical sensor is configured to detect a light signal comprising at least one of a reflected portion of the first and second light signals and a diffracted portion of the first and second light signals. The sensor apparatus also includes a processor configured to determine a concentration of smoke particles in the exhaust gas stream based on the detected light signal.

### CROSS-REFERENCE TO RELATED APPLICATIONS

This application claims priority under 35 USC § 119(e) of U.S. provisional patent application 63/478,570, the specification of which is hereby incorporated by reference.

### TECHNICAL FIELD

The technical field generally relates to solid fuel-burning appliances and, more particularly, to optical particle sensors for use with solid fuel-burning appliances. It also relates to a method for monitoring a concentration of smoke particles in an exhaust gas stream of a solid fuel burning appliance.

### BACKGROUND

Solid fuel-burning appliances, such as fireplaces, wood stoves, and the like, have been used in residential environments. In pre-industrial eras, it was common to provide an open hearth, with smoke rising through the room to a smoke hole in the roof. More recently, it is common to install a fireplace or wood stove near the side of a room, and to provide a duct or chimney to direct exhaust gasses out of the room.

In more modern solid fuel-burning appliances, one or more sensors may be provided to monitor the operation of the appliance. For example, the temperature within the combustion chamber may be displayed to a user. This may allow a user to make a more informed decision regarding the operation of the appliance. For example, a user may review the temperature and decide to add additional fuel to the combustion chamber and/or modify the air inlets.

Residential solid fuel-burning appliances are one of the main causes of pool air quality in winter in several cities of North America. According to Natural resources Canada (https://oee.rncan.gc.ca/corporate/statistics/neud/dpa/showTable.cfm?type=CP& sector=res&juris=qc&rn=14&year=2020&page=4), residential wood heating is responsible for 19.9% of fine particulate matter emissions. At least for environmental purposes, there is therefore a need to monitor and reduce the fine particulate matter emission in residential or light-commercial solid fuel-burning appliances.

### SUMMARY

The following introduction is provided to introduce the reader to the more detailed discussion to follow. The introduction is not intended to limit or define any claimed or as yet unclaimed invention. One or more inventions may reside in any combination or sub combination of the elements or process steps disclosed in any part of this document including its claims and figures.

One important performance metric of a solid fuel-burning appliance is the exhaust emitted from the combustion chamber, which is often referred to as smoke. Accordingly, it may be desirable to monitor and/or quantify the smoke produced during operation of a solid fuel-burning appliance.

For example, it may be desirable to monitor the smoke to determine and/or control the performance (e.g. combustion efficiency) of the solid fuel-burning appliance. Additionally, or alternatively, it may be desirable to monitor the smoke for safety reasons. Also, in some jurisdictions, there may be a requirement to limit the quantity and/or quality of smoke emitted.

Monitoring of smoke particles using optical detection methods is often employed in industrial settings. For example, solid fuel-burning power plants may be required to limit smoke emissions, which often requires logging data regarding spoke output. In industrial settings, it is common to use optical systems based on detecting opacity, or based on ‘transmissive’ or ‘forward’ light scattering. While industrial systems may be highly accurate and robust, they may not be considered appropriate for smaller solid fuel-burning appliances, such as those used in residential or light commercial (e.g. hotel, office lobby) settings.

Optical sensors and detection methods disclosed herein may be generally characterized as using ‘backwards’ light scattering as a basis for determining a concentration of smoke particles in an exhaust gas stream. Such a ‘backwards’ light scattering system may have one or more advantages. For example, in contrast to other optical systems, in the sensor apparatus disclosed herein the light source(s) and detector(s) are mounted on the same side a duct conveying the smoke to be monitored. This may reduce the cost of the apparatus, and/or simplify its installation. Additionally, or alternatively, such an arrangement may be characterized as being easier to clean and/or otherwise maintain. Additionally, or alternatively, such an arrangement may reduce or minimize the complexity of an initial alignment. Additionally, or alternatively, such an arrangement does not require a communication cable crossing a high-temperature zone. Additionally, or alternatively, such an arrangement may only have one portion of the apparatus that could require cooling.

Also, optical sensors and detection methods disclosed herein may emit and/or detect light at specified frequency or wavelength ranges. This may improve the accuracy of smoke particle detection. It may also allow for estimates of the composition of the smoke. For example, an estimate of a ratio of water vapour to other smoke particles may be determined.

Also, sensors and detection methods disclosed herein may detect and/or quantify smoke occurrence faster than methods that rely on a temperature sensor located inside the combustion chamber. This may facilitate improved control of combustion conditions.

Also, sensors and detection methods disclosed herein may be used in conjunction with temperature sensor(s) located inside (or proximate to) the combustion chamber. For example, an optical smoke sensor may be used to confirm that a pre-determined target temperature for the combustion chamber is adequate for the fuel inserted in the combustion chamber.

In accordance with one broad aspect, there is provided a sensor apparatus for determining a concentration of smoke particles in an exhaust gas stream flowing through a duct. The apparatus comprises: an optical sensor; a lens positioned in a detection light path extending between the optical sensor and a target area of the exhaust gas stream; a first light source positioned adjacent to a first side of the optical sensor, the first light source being configured to emit a first light signal towards the target area along a first light path extending between the first light source and the target area; a second light source positioned adjacent to a second side of the optical sensor, the second light source being configured to emit a second light signal towards the target area along a second light path extending between the second light source and the target area; and a processor. The optical sensor is configured to detect a light signal from the target area. The detected light signal comprises at least one of a reflected portion of the first and second light signals and a diffracted portion of the first and second light signals. The processor is configured to determine a concentration of smoke particles in the exhaust gas stream based on the detected light signal.

In some embodiments, the target area is positioned within the duct and at least one of the first light path and the second light path passes through a viewing port defined in a peripheral of the duct.

In some embodiments, the sensor apparatus further comprises a temperature sensor operatively connected to the processor. The processor can be configured to compensate for a variation in sensitivity of the optical sensor as a function of temperature when determining the concentration of smoke particles. The processor can be configured to adjust a supply of electrical current to at least one of the first light source and the second light source to compensate for a variation in output illuminance of the first and second light sources as a function of temperature. The processor can be configured to compare a measured temperature to a predetermined maximum temperature and, if the measured temperature exceeds the predetermined maximum temperature, the processor can trigger at least one of a shut down of the apparatus and a transmission of an alert signal.

In some embodiments, a portion of the detection light path between the lens and the optical sensor is defined by a sealed tunnel.

In some embodiments, the apparatus further comprises an optical cover plate that overlies the optical sensor, the first light source, and the second light source. The optical cover plate can comprise: a first tunnel defining a portion of the first light path; a second tunnel defining a portion of the second light path; and a third tunnel defining a portion of the detection light path.

In some embodiments, the detection light path is straight, and the first light path intersects the target area at an angle to the detection light path of between about 5° to about 30°.

In some embodiments, the first light path intersects the target area at an angle to the detection light path of about 16°.

In some embodiments, at least one of the first light source and the second light source comprises a light emitting diode.

In some embodiments, the first light source is configured to emit the first light signal at an illuminance sufficient to provide a light signal of between about 0.01 lux and about 2 lux to the optical sensor.

In some embodiments, the first light source is configured to emit the first light signal as white light.

In some embodiments, the first light source is configured to emit the first light signal at one of at least two wavelength ranges.

In some embodiments, a first one of the of at least two wavelength ranges is white light, and a second one of the at least two wavelength at least partially overlaps the first one of the at least two wavelength ranges.

In some embodiments, the optical detector is configured to detect light from about 350 nm to about 1050 nm.

In some embodiments, the optical detector is configured to detect light from about 500 nm to about 850 nm.

In some embodiments, the apparatus further comprises a bandpass filter for limiting a bandwidth of an output signal from the optical sensor.

In some embodiments, the optical detector comprises a first photodiode sensitive to a first wavelength range and a second photodiode sensitive to a second wavelength range that is different from the first wavelength range.

In some embodiments, the first and second wavelength ranges at least partially overlap.

In some embodiments, the processor is configured to: direct the first light source to emit the first light signal at an initial wavelength range; receive a first signal from the optical sensor indicative of the detected light signal while the first light signal at the initial wavelength range is being emitted; direct at least one of: the first light source to emit the first light signal at a subsequent wavelength range, and the second light source to emit the second light signal; receive a second signal from the optical sensor indicative of the detected light signal while the at least one first light signal at the subsequent wavelength range and the second light signal is being emitted; and determine the concentration of smoke particles in the exhaust gas stream based on the first received signal and the second received signal.

In some embodiments, the apparatus further comprises an instrument housing, and the optical sensor, the lens, the first light source, the second light source, and the processor are positioned in the instrument housing.

In some embodiments, the sensor apparatus further comprises an insulated mount having a first face, a second face opposite to the first face, and an optical aperture extending through an insulation panel from the first face to the second face. The first face is configured to abut the duct with the optical aperture surrounding the viewing port. When the instrument housing is mounted to the second face, the detection light path, the first light path, and the second light path each pass through the optical aperture.

In some embodiments, the target area is positioned within the duct, and the detection light path passes through a viewing port defined in the duct.

In accordance with still another broad aspect, there is provided a solid fuel-burning appliance comprising: a combustion chamber; an exhaust duct in fluid communication with the combustion chamber for conveying smoke from the combustion chamber; and a sensor apparatus as defined above and positioned in alignment with a viewing port defined in the exhaust duct.

In some embodiments, the solid fuel-burning appliance further comprises: an air inlet duct in fluid communication with the combustion chamber via at least one flow restricting device; and a controller. The sensor apparatus can be configured to transmit a determined concentration of smoke particles in an exhaust gas stream flowing through the exhaust duct to the controller. The controller can be configured to adjust a flow rate of air into the combustion chamber by adjusting the at least one flow restricting device based on the determined concentration of smoke particles

In accordance with another broad aspect, there is provided a solid fuel-burning appliance comprising: a combustion chamber; an exhaust duct in fluid communication with the combustion chamber for conveying smoke from the combustion chamber; a sensor apparatus positioned in alignment with a viewing port defined in the exhaust duct, a controller; and an air inlet conduit in fluid communication with the combustion chamber via at least one flow restricting device; wherein the sensor apparatus is configured to transmit a determined concentration of smoke particles in an exhaust gas stream flowing through the exhaust duct to the controller, and wherein the controller is configured to adjust a flow rate of air into the combustion chamber by adjusting the at least one flow restricting device based on the determined concentration of smoke particles.

In some embodiments, the solid fuel-burning appliance is a wood burning stove.

In accordance with a further broad aspect, there is provided a sensor apparatus for determining a concentration of smoke particles in an exhaust gas stream flowing through a duct having a viewing port defined therein. The apparatus comprises: a light source configured to emit light towards a target area of the exhaust gas stream and along a light path extending between the light source and the target area, the light source being configured to emit light in at least two wavelength ranges and the target area being positioned within the duct; an optical sensor configured to detect a light signal from the target area, the detected light signal comprising at least one of a reflected portion of the emitted light and a diffracted portion of the emitted light; a detection light path extending between the optical sensor and the target area of the exhaust gas stream and passing through the viewing port of the duct; and a processor. The process is configured to: direct the light source to emit an initial light signal at an initial wavelength range; receive an initial signal from the optical sensor indicative of the detected light signal while the initial light signal is being emitted; direct the light source to emit a subsequent light signal at a subsequent wavelength range; receive a subsequent signal from the optical sensor indicative of the detected light signal while the subsequent light signal is being emitted; and determine at least one of a concentration of smoke particles in the exhaust gas stream and at least a partial composition of smoke particles in the exhaust gas stream based on the initial received signal and the subsequent received signal.

In accordance with another broad aspect, there is provided a sensor apparatus for determining a concentration of smoke particles in an exhaust gas stream flowing through a duct, the apparatus comprising: an optical sensor; a detection light path extending between the optical sensor and a target area of the exhaust gas stream; a light source positioned adjacent to the optical sensor, the light source being configured to emit light towards the target area along a light path extending between the light source and the target area; and a processor; wherein the optical sensor is configured to detect a light signal from the target area, the detected light signal comprising at least one of a reflected portion of the emitted light and a diffracted portion of the emitted light, and wherein the processor is configured to: direct the light source to emit an initial light signal at an initial wavelength range; receive an initial signal from the optical sensor indicative of the detected light signal while the initial light signal is being emitted; direct the light source to emit a subsequent light signal at a subsequent wavelength range; receive a subsequent signal from the optical sensor indicative of the detected light signal while the subsequent light signal is being emitted; and determine at least one of a concentration of smoke particles in the exhaust gas stream and a composition of smoke particles in the exhaust gas stream based on the initial received signal and the subsequent received signal.

In some embodiments, the target area is positioned within the duct, and the detection light path passes through a viewing port defined in the duct.

In accordance with another broad aspect, there is provided a combustion particle sensor apparatus for monitoring a concentration of smoke particles in an exhaust gas stream flowing through a duct. The apparatus comprises: an instrument housing having a duct mounting face superposable against the duct with an optical aperture extending therethrough; an optical sensor located inside the instrument housing and being in light communication with the optical aperture; a first light source configured to emit a first light signal along a first light path extending to the optical aperture; and a processor in data communication with the optical sensor and configured to determine the concentration of smoke particles using at least one of a reflected portion and a diffracted portion of the first light signal emitted by the first light source. In an embodiment, the combustion particle sensor apparatus further comprises a second light source configured to emit a second light signal along a second light path extending to the optical aperture, the second light path intersecting with the first light path outside of the instrument housing. The processor can determine the concentration of smoke particles using at least one of the reflected portion and the diffracted portion of the first and second light signals emitted by the first and the second light sources

In accordance with another broad aspect, there is provided a solid fuel burning appliance comprising: a combustion apparatus having a combustion chamber defined therein; an exhaust duct extending from the combustion apparatus and in fluid communication with the combustion chamber, the exhaust duct having a viewing port extending therethrough; and a combustion particle sensor apparatus as detailed above and mounted to the exhaust duct along a first quarter of a length of the exhaust conduit, closer to the combustion chamber.

In accordance with another broad aspect, there is provided a method for monitoring a concentration of smoke particles in an exhaust gas stream flowing through an exhaust conduit of a solid fuel burning appliance. The method comprises: emitting two light signals from light sources located outside of the exhaust duct along two light paths that intersect inside the exhaust duct; detecting at least one of a reflected portion and a diffracted portion of the two light signals using an optical sensor located outside of the exhaust duct; and correlating the detected light signal to the concentration of smoke particles in the exhaust gas stream.

In an embodiment, emitting of the two light signals and detecting of the at least one of the reflected portion and the diffracted portion of the two light signals comprise: emitting a first one of the two light signals from at least one of the light sources at an initial wavelength range; receiving a first signal from the optical sensor indicative of the detected light signal while the first one of the light signals at the initial wavelength range is being emitted; emitting a second one of the two light signals from at least one of the light sources at a subsequent wavelength range, and receiving a second signal from the optical sensor indicative of the detected light signal while the second one of the light signals at the subsequent wavelength range is being emitted

It will be appreciated by a person skilled in the art that an apparatus or method disclosed herein may embody any one or more of the features contained herein and that the features may be used in any particular combination or sub-combination.

### BRIEF DESCRIPTION OF THE DRAWINGS

For a better understanding of the described embodiments and to show more clearly how they may be carried into effect, reference will now be made, by way of example, to the accompanying drawings in which:

FIG. 1 is a perspective view of a sensor apparatus, according to one embodiment, with the apparatus secured to a duct section of a solid fuel-burning appliance;

FIG. 2 is a front plan view of the sensor apparatus and duct of FIG. 1;

FIG. 3 is a section view of the sensor apparatus and duct of FIG. 1, taken along line 3-3 in FIG. 2;

FIG. 4 is a section view of the sensor apparatus and a portion of the duct section of FIG. 1, taken along line 4-4 in FIG. 2, with an optional cooling fan omitted for clarity;

FIG. 5 is a perspective view of a circuit board, a light and lens holder, and a light and lens cover of the sensor apparatus of FIG. 1, in accordance with an embodiment;

FIG. 6 is a partially exploded perspective view of the circuit board, light and lens holder, and light and lens cover of FIG. 5;

FIG. 7 is a top plan view of the circuit board, light and lens holder, and light and lens cover of FIG. 5;

FIG. 8 is a section view of the circuit board, light and lens holder, and light and lens cover of FIG. 5, taken along line 8-8 in FIG. 7;

FIG. 9 is a top section view of a sensor apparatus according to another embodiment, with the apparatus secured to a duct section of a solid fuel-burning appliance;

FIG. 10 is a perspective section view of the sensor apparatus of FIG. 9; and

FIG. 11 is a schematic view of a solid-fuel burning system that includes a solid fuel-burning appliance and a sensor apparatus;

The drawings included herewith are for illustrating various examples of apparatus and methods of the teaching of the present specification and are not intended to limit the scope of what is taught in any way.

### DETAILED DESCRIPTION

Various methods and systems are described below to provide example embodiments and implementations of the technology. The technology includes methods and systems that facilitate the remediation of a tailings pond.

While the systems and methods disclosed herein are described specifically in relation to solid-fuel burning stoves, such as wood-burning stoves and fireplaces, for residential and use, it will be appreciated that the sensor apparatus and methods may alternatively be used with other appliances that include a combustion chamber, and/or in commercial applications.

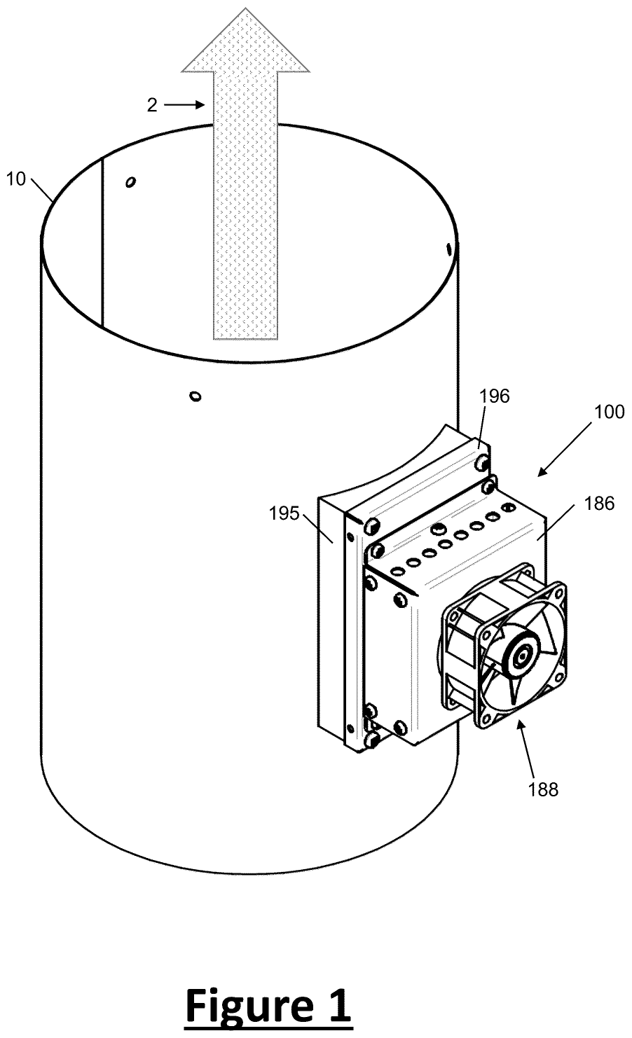

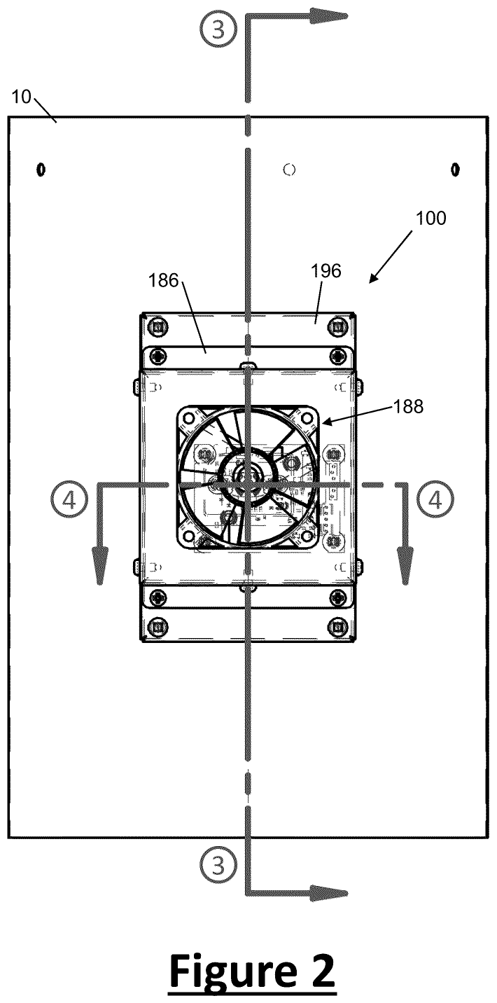

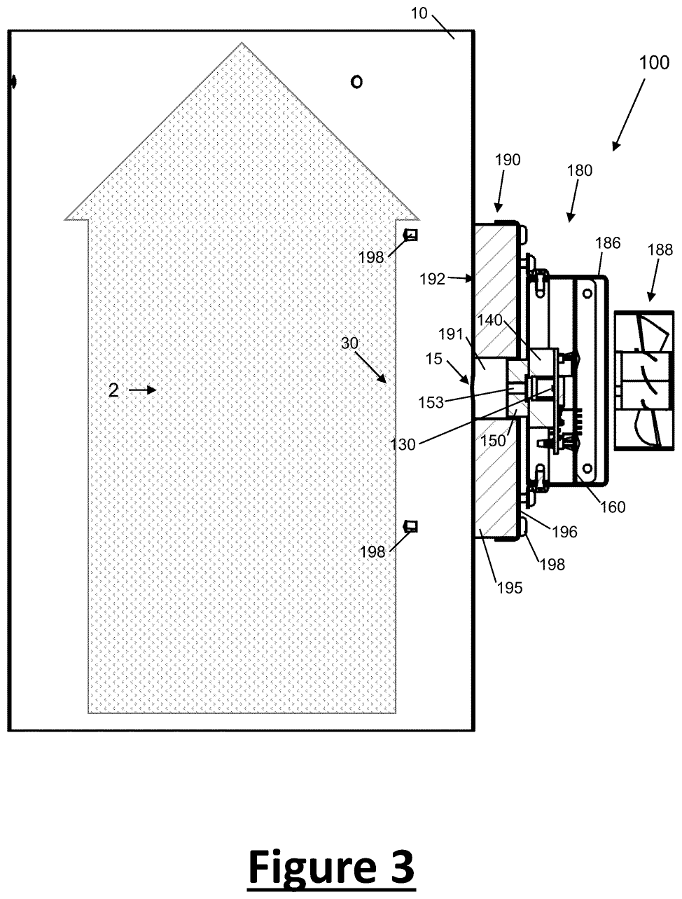

FIGS. 1 to 4 illustrate an example of a sensor apparatus, referred to generally as 100, secured to a portion of a duct 10. Duct 10 is configured to convey a fluid stream 2 (which may also be referred to as an exhaust gas stream) from a combustion chamber towards an exhaust outlet. For example, the combustion chamber may be part of a solid fuel-burning appliance, such as a fireplace or wood stove, and the duct 10 may lead to and/or form part of a chimney. When the combustion chamber is in use, the fluid stream 2 conveyed by duct 10 will contain exhaust particles and gases, which may also be referred to as smoke.

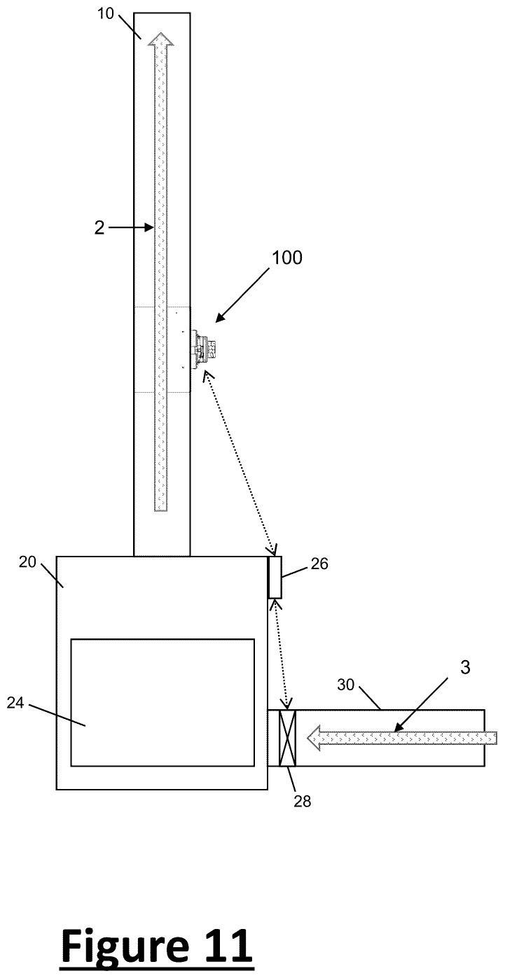

Sensor apparatus 100 may be positioned anywhere along a duct that conveys exhaust gas from the combustion chamber. For example, with reference to FIG. 11, sensor apparatus 100 may be positioned within the first 5 feet of the ducting extending from a solid fuel-burning appliance. In an embodiment, sensor apparatus 100 may be positioned between about 1 foot and 2 feet above a chimney connector ring of the solid fuel-burning appliance. It may also be installed further than 5 feet from the solid fuel-burning appliance. In an embodiment, the sensor apparatus 100 is positioned in a first quarter of a length of the chimney, closer to the solid fuel burning apparatus and its combustion chamber.

Duct 10 may be an existing duct, or ducting installed during installation of the solid fuel-burning appliance. Duct 10 may be a single-wall conduit, a double-walled conduit without insulation, a double-walled conduit with insulation, a triple-walled conduit, or any other suitable conduit for conveying an exhaust gas stream.

Duct 10 may have any suitable size or geometry. For example, duct 10 may have a diameter of between about 6 inches to about 12 inches, or be larger or smaller.

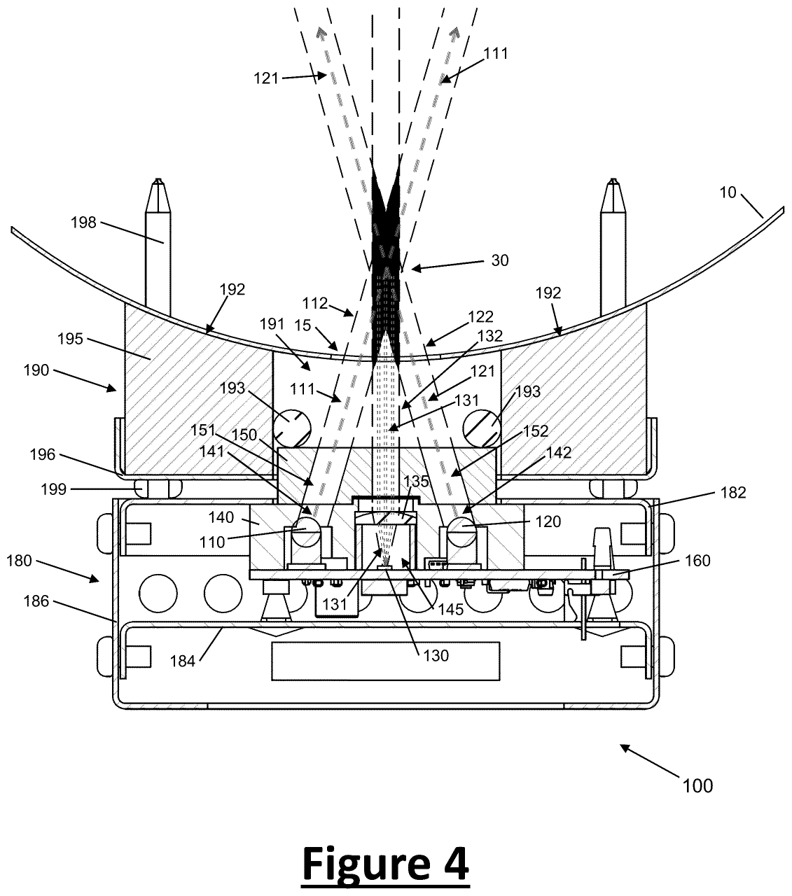

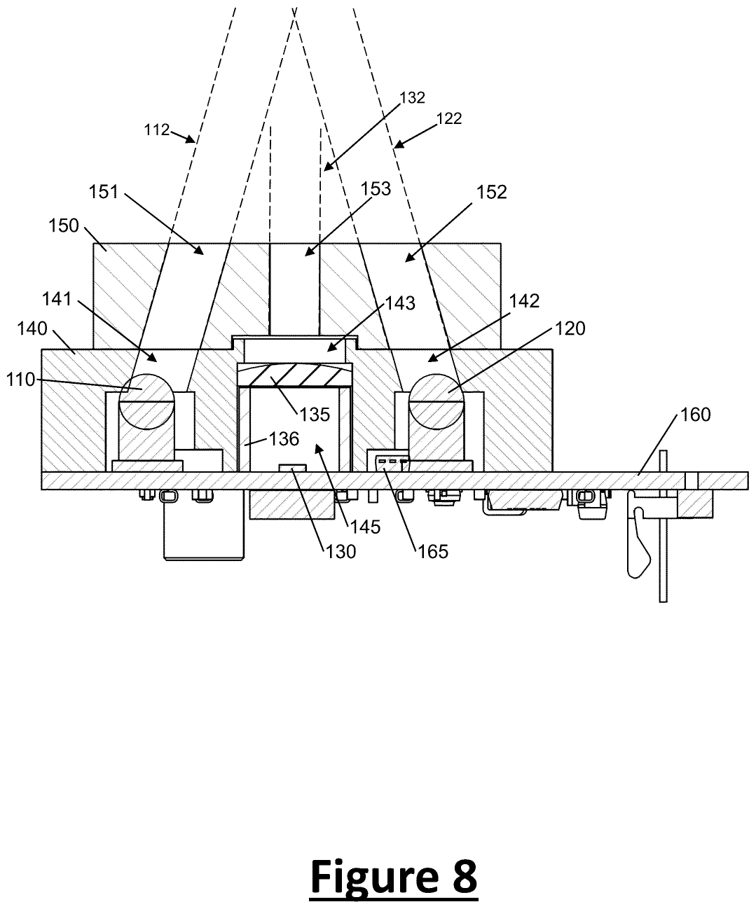

With reference to FIGS. 4 and 8, sensor apparatus 100 includes an optical sensor 130, a first light source 110, and a second light source 120. As illustrated in FIG. 4, the first light source 110 is configured to emit light 111 along a first light path 112 through a viewing port 15 in duct 10 and towards a target area 30 within duct 10, and the second light source 120 is configured to emit light 121 along a second light path 122 through viewing port 15 and towards target area 30, located inside the duct 10 and where both light paths 112, 122 intersect. First and second light source 110, 120 may be operated to emit light concurrently, sequentially, or alternately.

Light sources 110, 120 may be used to illuminate the target area 30 in a desired manner. For example, one or both of the light sources 110, 120 may be configured to selectively emit light within a specified wavelength or frequency range. For example, one or both of light sources 110, 120 may be configured to emit ‘white’ light (e.g. across at least most or all of the visible spectrum of about 380 nm to about 800 nm), or to emit light across a broader wavelength or frequency range, e.g. from about 350 nm to about 1050 nm. In examples where two or more light sources are provided, they may each be configured to emit light within the same (or substantially the same) wavelength or frequency range.

Alternatively, two or more light sources may each be configured to emit light within different, overlapping wavelength or frequency ranges. For example, a first light source may be configured to emit light of about 350 nm to about 800 nm, and a second light source may be configured to emit light of about 750 nm to about 1050 nm).

Alternatively, two or more light sources may each be configured to emit light within different, non-overlapping wavelength or frequency ranges. For example, a first light source may be configured to emit light of about 450 nm to about 485 nm, and a second light source may be configured to emit light of about 625 nm to about 725 nm).

Additionally, or alternatively, one or both of light sources 110, 120 may be configured to selectively emit light at one or more specific wavelengths or frequencies. For example, one or both of light sources 110, 120 may be configured to emit ‘blue’ light (e.g. at one or more wavelengths of 450 nm to 485 nm). In examples where two or more light sources are provided, they may each be configured to emit light at the same (or substantially the same) specific wavelength or frequency, or they may each be configured to emit light at different specific wavelengths or frequencies. The different specific wavelengths or frequencies may be overlapping or non-overlapping.

As another example, one or both of the light sources 110, 120 may be configured to selectively emit light at a specified illuminance. For example, one or both of light sources 110, 120 may be configured to provide an illuminance sufficient to provide a light signal of between about 0.01 lux to 2 lux to light sensor 130. It will be appreciated that the illuminance of the light sources 110, 120 required to provide sufficient illuminance to the light sensor will vary based on a number of factors, e.g. the geometry of the duct 10, the distance between the light sources 110, 120 and the target area 30, the distance between the target area 30 and the light sensor 130, etc.

Light sources 110, 120 may each include one or more light emitting diodes (LEDs). For example, LEDs such as those available from Cree, Inc. of Durham, NC, U.S.A. may be used in one or more embodiments.

It will be appreciated that the light output of some light sources (e.g. LEDs) may be dependent on the physical temperature of the light source. Optionally, sensor apparatus 100 may include a temperature sensor. In the illustrated example, temperature sensor 165 (FIG. 8) is provided proximate the second light source 120. Where a temperature sensor is provided, sensor apparatus 100 may be configured to adjust a supply of electrical current to one or both of light sources 110, 120 to compensate for a variation in output illuminance of the first and second light sources as a function of temperature.

Providing at least two light sources 110, 120 may have one or more advantages. For example, using two LEDs may allow for a much greater range of emitted light power as would be possible with a single LED. In this respect, the current (and therefore the power) of typical LEDs must generally be limited to avoid non-linear behaviour as temperature increases. By using two or more light sources in series, the electrical power required for each light source can be reduced by half while keeping the total light power substantially constant.

Returning to FIG. 4, when the first and/or second light source 110, 120 emit light 111, 121 towards target area 30, at least a portion of the light 111, 121 is reflected and/or diffracted by smoke particles in the exhaust gas stream passing through target area 30, and at least some of the reflected and/or diffracted light 131 is directed through viewing port 15 and towards the optical sensor 130.

Optical sensor 130 is configured to detect light signal 131 from target area 30, which includes reflected and/or diffracted portions of light 111 and/or 121. For example, optical sensor 130 may generate an output signal that is proportionate to the illuminance and/or wavelength of detected light 131.

Sensor apparatus 100 includes at least one processor (not shown), such as a logic chip, programmable logic controller (PLC), and the like. Such a processor (or processors) is configured to determine a concentration of smoke particles in target area 30 based on the detected light signal 131. For example, the processor(s) may correlate one or more output signals from optical sensor 130 to determine an estimated particle count. In an embodiment, the optical sensor 130 is used to monitor smoke particles smaller or equal to about 2.5 micrometers.

It will be appreciated that sensor apparatus 100 may include one or more signal processing components or circuits to facilitate the determination of a concentration of smoke particles. For example, one or more bandpass filters may be provided for limiting a bandwidth of an output signal from the optical sensor.

In some examples, sensor apparatus 100 may be configured to determine one or more other properties of the smoke passing through target area 30. For example, water vapour typically absorbs photons having a wavelength of about 600 nm and 800 nm significantly more than photons of other wavelengths. Sensor apparatus 100 may be configured to direct one or both of light sources 110, 120 to initially emit light within such a wavelength range (e.g. at more or more wavelengths between 600 nm and 800 nm), and to direct one or both of light sources 110, 120 to subsequently emit light within a different wavelength range (e.g. at wavelengths between about 350 nm to about 1050 nm). By comparing output signals from optical sensor 130 during the initial illumination with output signals from optical sensor 130 during the subsequent illumination, sensor apparatus 100 may determine an estimated ratio of water vapour to other particles in the smoke.

Determining an estimated ratio of water vapour to other particles in the smoke may have one or more advantages. For example, such a water vapour ratio may be used to select a particular ‘combustion strategy’. If the water vapour ratio is relatively high (i.e. smoke is mainly caused by water vapor), this may imply that a higher flow rate of combustion air at a primary level of the combustible fuel (e.g. the base or lower portion of the fuel) is required in order to generate more heat, thereby accelerating evaporation of the combustible fuel). If the water vapour ratio is relatively low (i.e. smoke is mainly caused by particulate matter, which may be the case for very dry fuel), this may imply that a lower flow rate of combustion air at a primary level of the combustible fuel, and a higher flow rate of combustion air at a secondary level of the combustible fuel may be more appropriate.

In some examples, such a water vapour ratio may be used to provide guidance to a user of the appliance. For example, if the water vapour ratio is relatively high, a message may be provided on a display screen associated with the sensor apparatus 100 and/or the appliance (not shown) to indicate that ‘Your fuel is wet, use dryer fuel or split you fuel into smaller pieces’. As another example, if the water vapour ratio is relatively low, a message may be provided to indicate that ‘Your fuel is very dry, use bigger wood piece(s) to reduce smoke’.

Optical sensor 130 may be any sensor capable of detecting light within desired frequency or wavelength ranges. For example, optical sensor 130 may include one or more photodiodes sensitive to light in a first wavelength range, and one or more photodiodes sensitive to light in a second wavelength range. For example, light sensors such as those available from ams-OSRAM AG of PremstAtten, Austria may be used in one or more embodiments.

It will be appreciated that the performance of certain optical sensors may be dependent on the physical temperature of the sensor. Optionally, where sensor apparatus 100 includes a temperature sensor, sensor apparatus 100 may be configured to adjust a supply of electrical current to optical sensor 130 to compensate for a variation in detection performance as a function of temperature. Alternatively, or additionally, sensor apparatus 100 may be configured to re-calculate an output (without modifying the input signal) to compensate for a variation in detection performance as a function of temperature.

Optionally, one or more lenses may be provided in a light path 132 that extends between the optical sensor 130 and target area 30. In the illustrated example, lens 135 is provided to focus light 131 towards optical sensor 130.

Referring to FIG. 8, in the illustrated example lens 135 is mounted on a spacer ring 136 that surrounds optical sensor 130. Such an arrangement may advantageously provide a sealed tunnel 145 surrounding optical sensor 130, which may inhibit or prevent smoke particles (or other particles) from dirtying optical sensor 130.

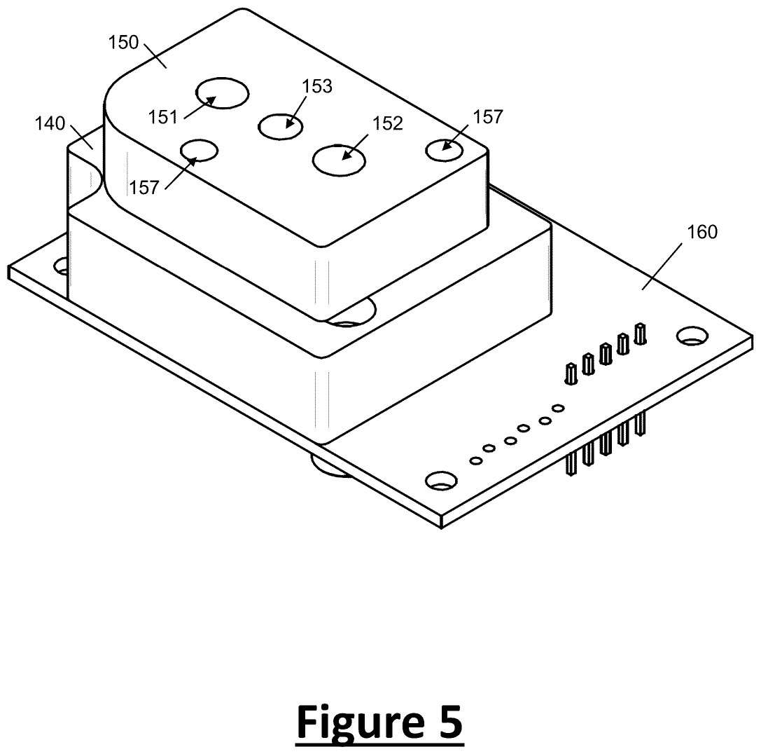

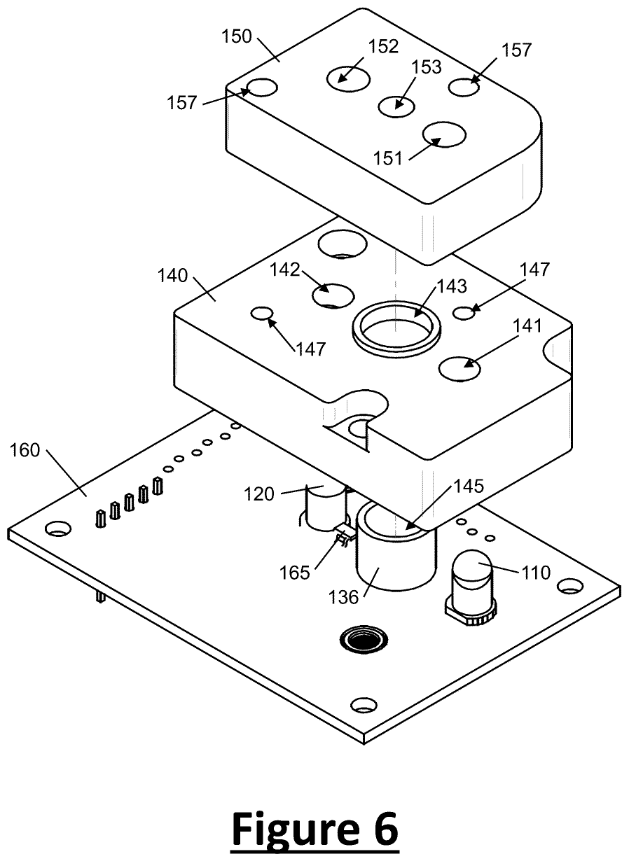

With reference to FIGS. 5 to 8, in the illustrated example optical sensor 130, first light source 110, and second light source 120 are mounted on a circuit board 160.

In the illustrated example, sensor apparatus 100 includes a holder 140 that surrounds first light source 110, second light source 120, and optical sensor 130. Preferably, holder 140 optically isolates optical sensor 130 from light sources 110, 120, to inhibit or prevent light emitted by the light sources from ‘leaking’ onto sensor 130. In the illustrated example, holder 140 includes a first conduit or tunnel 141 that forms a portion of first light path 112, a second conduit or tunnel 142 that forms a portion of second light path 122, and a third conduit or tunnel 143 that forms a portion of detection light path 132. Holder 140 may be made from any suitable opaque material, such as nylon, acrylonitrile butadiene styrene (ABS), and the like. In an embodiment, holder 140 is formed from a material with low thermal conductivity (e.g. a non-metallic material) to reduce or minimize heat conduction to circuit board 160. Also, the surfaces of holder 140 can be black, with a non-reflective finish (e.g. a matte finish) to reduce or minimize internal light reflection.

Also, in the illustrated example, sensor apparatus 100 includes an optical cover plate 150 that is mounted to holder 140. Preferably, cover plate 150 is removably mounted to holder 140 (e.g. via screws inserted in holes 157, 147) to facilitate e.g. cleaning or replacing lens 135. Additionally, or alternatively, holder 140 may be removably mounted to circuit board 160, to facilitate cleaning or replacing light sources 110, 120.

In the illustrated example, cover plate 150 includes a first conduit or tunnel 151 that forms a portion of first light path 112 and is in light communication and/or aligned with first conduit or tunnel 141, a second conduit or tunnel 152 that forms a portion of second light path 122 and is in light communication and/or aligned with second conduit or tunnel 142, and a third conduit or tunnel 153 that forms a portion of detection light path 132 and is in light communication and/or aligned with third conduit or tunnel 143. Cover plate 150 may be made from any suitable material, such as nylon, acrylonitrile butadiene styrene (ABS), and the like. In an embodiment, cover plate 150 is formed from a material with low thermal conductivity to reduce or minimize heat conduction to holder 140. Also, the surfaces of cover plate 150 can be black, with a non-reflective finish, to reduce or minimize internal light reflection.

Returning to FIG. 4, in the illustrated example each of light paths 112, 122, and 132 are generally straight. An advantage of such a configuration is that, aside from lens 135, no other transmissive optical devices are required to direct light from light sources 110, 120 to target area 30 and/or from target area 30 to optical sensor 130.

First and second light paths 112 and 122 (and light 111, 121 emitted along these light paths) are oriented at an angle to detection light path 132. Such an arrangement may have one or more advantages. For example, such an arrangement allows the emitted light 111, 121 to be concentrated in the area of interest 30 (i.e. where smoke is expected to be), while avoiding signal overlap in other portions of duct 10. Additionally, or alternatively, such an arrangement may reduce or minimize light reflected by the interior surface of duct 10 from reaching optical sensor 130. This may improve the precision and/or accuracy of the smoke particle count.

In the illustrated example, first light path 112 is oriented at an angle of about 16° to detection light path 132, and second light path 122 is also oriented at an angle of about 16° to detection light path 132. It will be appreciated that light paths 112 and 122 may be oriented at different angles to detection light path 132 in some embodiments. For example, the angle between first light path 112 and detection light path 132 may vary based on the diameter of duct 10, and/or based on a desired location of target area 30 within duct 10. For example, the first light path may intersect the target area at an angle to the detection light path of between about 5° to about 30°.

Sensor apparatus 100 may be generally characterized as using ‘backwards’ light scattering as a basis for determining a concentration of smoke particles in an exhaust gas stream. This operating principle may be contrasted with other optical particle measurement systems, such as ‘transmissive’ or ‘forward’ light scattering systems. Such a ‘backwards’ light scattering system may have one or more advantages. For example, in contrast to other optical systems, in sensor apparatus 100 the light source(s) and detector(s) are mounted on the same side of duct 10.

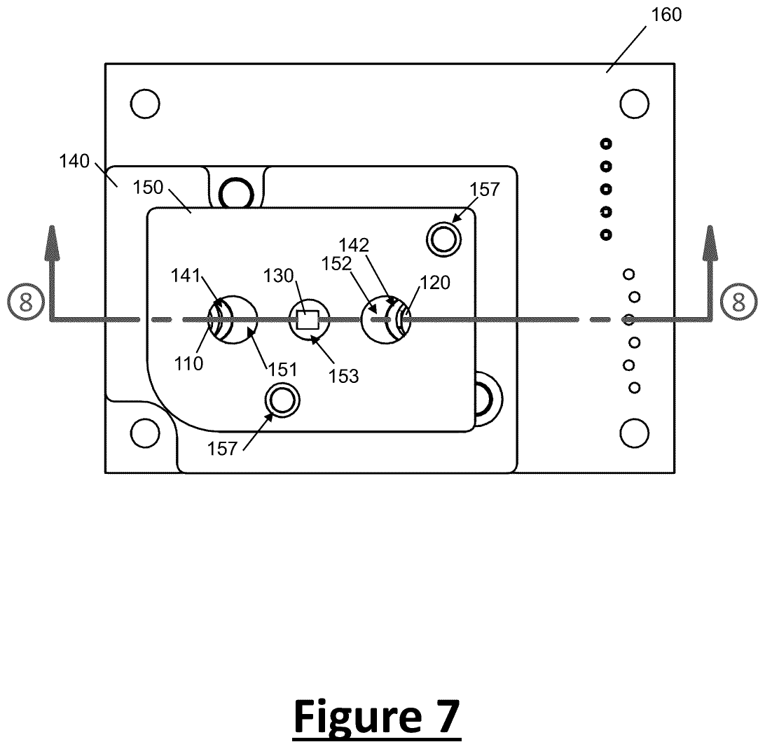

With reference to FIG. 7, the illustrated example, first light source 110, second light source 120, and optical sensor 130 are positioned in a generally straight line, with optical sensor positioned midway between the first and second light sources 110, 120. It will be appreciated that the light sources 110, 120 and sensor 130 may be positioned in one or more other relative arrangements. For example, light sources 110, 120 and sensor 130 may be arranged in a generally triangular arrangement, with one of 110, 120, and 130 positioned at each vertex. As another example, if three light sources are used, the light sources may be arranged in a generally triangular arrangement with a light source at each vertex, and with sensor 130 positioned at the centroid of the triangle.

With reference to FIGS. 1 to 4, sensor apparatus 100 includes an instrument housing, referred to generally as 180. In the illustrated example, instrument housing 180 includes a back plate 182, an intermediate plate 184, and a cover plate 186. Plates 182, 184, 186 may be made from any suitable material, such as plastic or bent sheet metal.

In the illustrated example, sensor apparatus 100 also includes an insulated mount, referred to generally as 190. In the illustrated example, insulated mount 190 includes a thermal insulation panel 195 having a first face 192 (or duct mounting face) and a mounting plate 196 defining a second face. First face 192 is shaped to abut the outer surface of duct 10, and the face of mounting plate 196 is substantially planar. An optical aperture 191 extends through the insulation panel 195. Insulation panel 195 may be made from a ceramic, or another suitable thermally insulative material.

Insulated mount 190 may be secured to duct 10 using any suitable method. In the illustrated example, four screws 198 are used to secure insulated mount 190 to duct 10. Insulated mount 190 is secured to duct 10 with optical aperture 191 surrounding viewing port 15.

Instrument housing 180 may be secured to insulated mount 190 using any suitable method. In the illustrated example, four screws are used to secure insulated mount 190 to duct 10.

Optionally, one or more spacers may be provided to further thermally isolate instrument housing 180 from insulated mount 190 (and therefore from duct 10). In the illustrated example, spacers 199 are provided between mounting plate 196 of insulated mount 190 and back plate 182 of instrument housing 180.

With reference to FIGS. 1 to 3, sensor apparatus 100 may include an external fan 188 to generate a flow of air over circuit board 160. It will be appreciated that in some embodiments, an external fan may not be necessary, as the ambient air may provide sufficient heat transfer to maintain the instruments of apparatus 100 within an acceptable temperature range.

In some situations, e.g. where sensor apparatus 100 is to be installed in an unventilated, relatively confined space, additional ducting may be provided to direct a flow of relatively cool air over at least instrument housing 180.

Optionally, one or more air-to-air or air-to-water heat exchangers may be provided to improve heat transfer (cooling) of one or more components mounted to circuit board 160. For example, heat exchanger(s) may be provided to cool the one or more light sources, as the output of the light sources may be temperature dependent. Additionally, or alternatively, heat exchanger(s) may be provided to cool the optical sensor 130, and/or other components (e.g. one or more processors, microchips, and/or PLCs)

Returning to FIG. 4, sensor apparatus 100 may have one or more features to inhibit or prevent smoke from duct 10 from traveling to and/or occluding first light source 110, second light source 120, and/or optical sensor 130. For example, sensor apparatus 100 may define one or more chambers to inhibit or prevent airflow. In the illustrated example, optical aperture 191 acts as a transition chamber to inhibit or prevent dust or smoke from entering tunnels 151, 152, or 153.

In the illustrated example, a gasket 193 is provided to inhibit or prevent airflow between the interior of aperture 191 and optical cover plate 150. Also, as noted above, lens 135 and spacer ring 136 provide a sealed tunnel 145 surrounding optical sensor 130. In such an arrangement, there may be little or no airflow into or out of a chamber defined by the interior of aperture 191, optical cover plate 150, and gasket 193. This may, for example, inhibit or prevent smoke backflow into sensor apparatus 100 when the duct 10 is cold (e.g. when the combustion chamber upstream of sensor apparatus 100 is not in use). Additionally, gasket 193 may inhibit or prevent smoke from entering the room in which sensor apparatus 100 is located.

As discussed above, sensor apparatus 100 is configured to determine a concentration of smoke particles an exhaust gas stream 2 based on a detected light signal 131 from target area 30. For example, sensor apparatus 100 may correlate one or more output signals from optical sensor 130 to determine an estimated particle count. For example, sensor apparatus 100 may estimate particle content expressed as grams per hour (g/h), grams per cubic meter (g/m³), or as other suitable unit(s).

Optionally, sensor apparatus 100 may be configured to monitor target area 30 to determine a cumulative particle count. For example, sensor apparatus 100 may continuously or periodically direct one or both of light sources 110, 120 to emit light to illuminate the target area 30 and to determine a concentration of smoke particles based on a concurrently detected light signal 131. Sensor apparatus 100 may also be configured to integrate the determined concentrations over the time period during which thy were determined to estimate a cumulative amount of smoke particles that have passed through duct 10 during the time period. Such a cumulative particle count may be used to estimate e.g. when light sources 110, 120 may require cleaning.

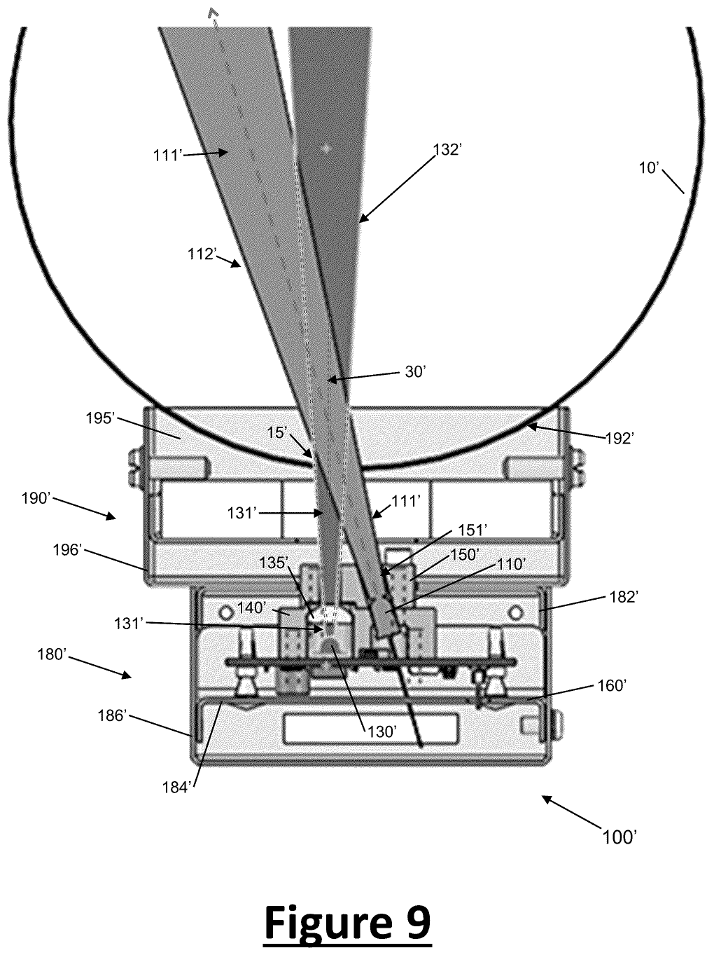

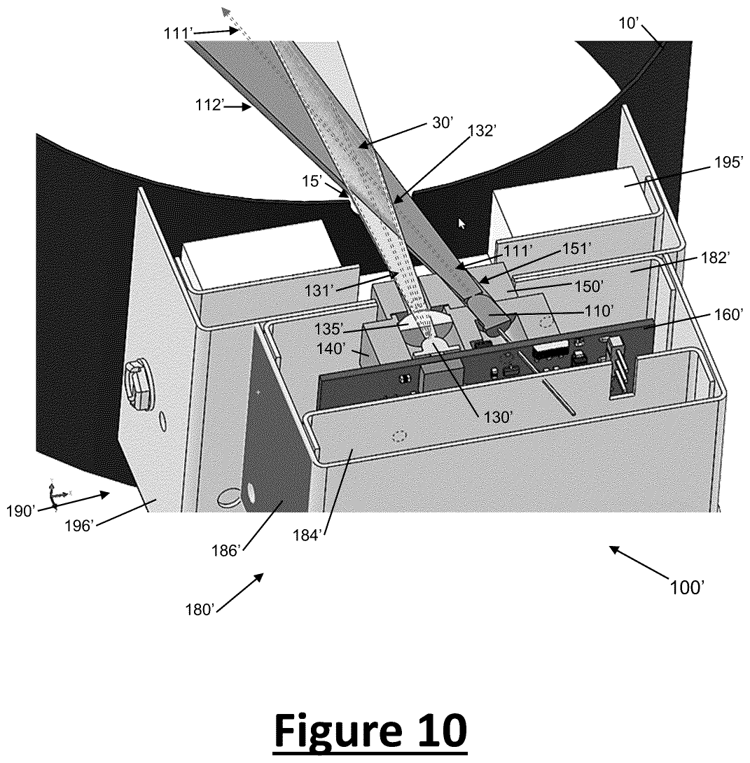

FIGS. 9 and 10 illustrate another example of a sensor apparatus 100′. Elements of sensor apparatus 100′ that have a similar function and/or structure to those in sensor apparatus 100 have been referred to using similar element numbering, and may not be discussed further.

In this example, sensor apparatus 100′ includes only one light source 110′, and optical sensor 130 is configured to detect light signal 131′ from target area 30′, which includes reflected and/or diffracted portions of light 111′.

FIG. 11 schematically illustrates a system that includes sensor apparatus 100. In the illustrated example, a solid fuel-burning appliance (e.g. a wood stove) 20 has a combustion chamber 24 in which solid fuel (e.g. wood, pellets, solid waste material, biomass, etc.) may be burned to generate heat and smoke. A duct 10 conveys smoke 2 (or exhaust air) from combustion chamber 24 to an exhaust outlet, such as a chimney. A duct 30 conveys inlet air 3 to combustion chamber 24 to provide oxygen to support the combustion of solid fuel.

In the illustrated example, a controller 26 is provided to monitor one or more aspects of the combustion within combustion chamber 24. For example, controller 26 may be configured to detect at least one of a temperature within combustion chamber 24, an outlet temperature of smoke 2, an ambient temperature of the room in which the stove 20 is positioned, a flow rate of inlet air 3, and a temperature of inlet air 3. For example, controller 26 may receive a smoke particle count from sensor apparatus 100.

Controller 26 may also be configured to control one or more aspects of the combustion within combustion chamber 24. In some examples, controller 26 may be configured adjust a flow restricting device 28 (such as louvres, a valve, a fan, and the like) to adjust a flow rate of air into the combustion chamber. For example, controller 26 may be configured to adjust flow restricting device 28 based on a determined concentration of smoke particles received from sensor apparatus 100.

For example, in response to an increased concentration of smoke particles in exhaust 2—which may be indicative of incomplete combustion within combustion chamber 24—controller 26 may adjust flow restricting device 28 to increase a flow rate of air into combustion chamber 24, in order to provide more oxygen to support combustion.

As another example, in response to an increased concentration of smoke particles in exhaust air 2 and a relatively high (or increased) temperature within the combustion chamber—which may be indicative of rapid combustion within combustion chamber 24—controller 26 may adjust flow restricting device 28 to decrease a flow rate of air into combustion chamber 24, in order to reduce the oxygen and thereby slow combustion.

In some examples, an appliance 20 may have two or more flow restricting devices 28, each being associated with a different combustion zone (e.g. one for a primary combustion zone, one for a secondary (and/or tertiary) combustion zone, one for a ‘window wash’ combustion zone). In response to an increased concentration of smoke particles in exhaust air 2, one or more flow restricting devices 28 may be adjusted to adjust combustion parameters in an effort to reduce the concentration of smoke particles. Optionally, if adjustment of flow restricting devices 28 is unsuccessful in reducing the smoke particle count, a message or alert may be conveyed to a user suggesting possible corrective action(s), e.g. stir the embers, add additional fuel, remove unburnt fuel, etc.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

As used herein, the wording “and/or” is intended to represent an inclusive—or. That is, “X and/or Y” is intended to mean X or Y or both, for example. As a further example, “X, Y, and/or Z” is intended to mean X or Y or Z or any combination thereof.

While the above description describes features of example embodiments, it will be appreciated that some features and/or functions of the described embodiments are susceptible to modification without departing from the spirit and principles of operation of the described embodiments. For example, the various characteristics which are described by means of the represented embodiments or examples may be selectively combined with each other. Accordingly, what has been described above is intended to be illustrative of the claimed concept and non-limiting. It will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

We claim:

1. A sensor apparatus for determining a concentration of smoke particles in an exhaust gas stream flowing through a duct, the apparatus comprising:
an optical sensor;
a lens positioned in a detection light path extending between the optical sensor and a target area of the exhaust gas stream;
a first light source positioned adjacent to a first side of the optical sensor, the first light source being configured to emit a first light signal towards the target area along a first light path extending between the first light source and the target area;
a second light source positioned adjacent to a second side of the optical sensor, the second light source being configured to emit a second light signal towards the target area along a second light path extending between the second light source and the target area; and
a processor;
wherein the optical sensor is configured to detect a light signal from the target area, the detected light signal comprising at least one of a reflected portion of the first and second light signals and a diffracted portion of the first and second light signals, and
wherein the processor is configured to determine a concentration of smoke particles in the exhaust gas stream based on the detected light signal.

2. The sensor apparatus of claim 1, wherein the target area is positioned within the duct and at least one of the first light path, the second light path, and the detection light path passes through a viewing port in the duct.

3. The sensor apparatus of claim 1, further comprising a temperature sensor operatively connected to the process, wherein the processor is configured to at least one of:
compensate for a variation in sensitivity of the optical sensor as a function of temperature when determining the concentration of smoke particles;
adjust a supply of electrical current to at least one of the first light source and the second light source to compensate for a variation in output illuminance of the first and second light sources as a function of temperature; and
compare a measured temperature to a predetermined maximum temperature and, if the measured temperature exceeds the predetermined maximum temperature, trigger at least one of a shut down of the apparatus and a transmission of an alert signal.

4. The sensor apparatus of claim 1, wherein a portion of the detection light path between the lens and the optical sensor is defined by a sealed tunnel and wherein the sensor apparatus further comprises an optical cover plate that overlies the optical sensor, the first light source, and the second light source, the optical cover plate comprising:
a first tunnel defining a portion of the first light path;
a second tunnel defining a portion of the second light path; and
a third tunnel defining a portion of the detection light path.

5. The sensor apparatus of claim 1, wherein the detection light path is straight, and wherein the first light path intersects the target area at an angle to the detection light path of between about 5° to about 30°.

6. The sensor apparatus of claim 1, wherein at least one of the first light source and the second light source comprises a light emitting diode.

7. The sensor apparatus of claim 1, wherein the first light source is configured to at least one of:
emit the first light signal at an illuminance sufficient to provide a light signal of between about 0.01 lux and about 2 lux to the optical sensor; and
emit the first light signal as white light.

8. The sensor apparatus of claim 1, wherein the optical detector is configured to detect light from about 350 nm to about 1050 nm.

9. The sensor apparatus of claim 1, wherein the optical detector comprises a first photodiode sensitive to a first wavelength range and a second photodiode sensitive to a second wavelength range that is different from the first wavelength range.

10. The sensor apparatus of claim 1, wherein the processor is configured to:
direct the first light source to emit the first light signal at an initial wavelength range;
receive a first signal from the optical sensor indicative of the detected light signal while the first light signal at the initial wavelength range is being emitted;
direct at least one of:
the first light source to emit the first light signal at a subsequent wavelength range, and
the second light source to emit the second light signal;
receive a second signal from the optical sensor indicative of the detected light signal while the at least one first light signal at the subsequent wavelength range and the second light signal is being emitted; and
determine the concentration of smoke particles in the exhaust gas stream based on the first received signal and the second received signal.

11. The sensor apparatus of claim 2, further comprising an instrument housing, wherein the optical sensor, the lens, the first light source, the second light source, and the processor are positioned in the instrument housing.

12. The sensor apparatus of claim 11, further comprising an insulated mount having a first face, a second face opposite to the first face, and an optical aperture extending through an insulation panel from the first face to the second face, wherein the first face is configured to abut the duct with the optical aperture surrounding the viewing port, and wherein when the instrument housing is mounted to the second face, the detection light path, the first light path, and the second light path each pass through the optical aperture.

13. A solid fuel-burning appliance comprising:
a combustion chamber;
an exhaust duct in fluid communication with the combustion chamber for conveying smoke from the combustion chamber; and
a sensor apparatus according to claim 1 positioned in alignment with a viewing port defined in the exhaust duct.

14. The solid fuel-burning appliance of claim 13, further comprising:
an air inlet duct in fluid communication with the combustion chamber via at least one flow restricting device; and
a controller; and
wherein the sensor apparatus is configured to transmit a determined concentration of smoke particles in an exhaust gas stream flowing through the exhaust duct to the controller, and
wherein the controller is configured to adjust a flow rate of air into the combustion chamber by adjusting the at least one flow restricting device based on the determined concentration of smoke particles.

15. A sensor apparatus for determining a concentration of smoke particles in an exhaust gas stream flowing through a duct having a viewing port defined therein, the apparatus comprising:
a light source configured to emit light towards a target area of the exhaust gas stream and along a light path extending between the light source and the target area, the light source being configured to emit light in at least two wavelength ranges and the target area being positioned within the duct;
an optical sensor configured to detect a light signal from the target area, the detected light signal comprising at least one of a reflected portion of the emitted light and a diffracted portion of the emitted light;
a detection light path extending between the optical sensor and the target area of the exhaust gas stream and passing through the viewing port of the duct; and
a processor configured to:
direct the light source to emit an initial light signal at an initial wavelength range;
receive an initial signal from the optical sensor indicative of the detected light signal while the initial light signal is being emitted;
direct the light source to emit a subsequent light signal at a subsequent wavelength range;
receive a subsequent signal from the optical sensor indicative of the detected light signal while the subsequent light signal is being emitted; and
determine at least one of a concentration of smoke particles in the exhaust gas stream and at least a partial composition of smoke particles in the exhaust gas stream based on the initial received signal and the subsequent received signal.

16. A combustion particle sensor apparatus for monitoring a concentration of smoke particles in an exhaust gas stream flowing through a duct, the apparatus comprising:
an instrument housing having a duct mounting face superposable against the duct with an optical aperture extending therethrough;
an optical sensor located inside the instrument housing and being in light communication with the optical aperture;
a first light source configured to emit a first light signal along a first light path extending to the optical aperture; and
a processor in data communication with the optical sensor and configured to determine the concentration of smoke particles using at least one of a reflected portion and a diffracted portion of the first light signal emitted by the first light source.

17. The combustion particle sensor apparatus of claim 16, further comprising a second light source configured to emit a second light signal along a second light path extending to the optical aperture, the second light path intersecting with the first light path outside of the instrument housing and wherein the processor determines the concentration of smoke particles using at least one of the reflected portion and the diffracted portion of the first and second light signals emitted by the first and the second light sources.

18. A solid fuel burning appliance comprising:
a combustion apparatus having a combustion chamber defined therein;
an exhaust duct extending from the combustion apparatus and in fluid communication with the combustion chamber, the exhaust duct having a viewing port extending therethrough; and
the combustion particle sensor apparatus of claim 1 mounted to the exhaust duct along a first quarter of a length of the exhaust duct, closer to the combustion chamber.

19. A method for monitoring a concentration of smoke particles in an exhaust gas stream flowing through an exhaust duct of a solid fuel burning appliance, the method comprising:
emitting two light signals from light sources located outside of the exhaust duct along two light paths that intersect inside the exhaust duct;
detecting at least one of a reflected portion and a diffracted portion of the two light signals using an optical sensor located outside of the exhaust duct; and
correlating the detected light signal to the concentration of smoke particles in the exhaust gas stream.

20. The method of claim 19, wherein the emitting of the two light signals and the detecting of the at least one of the reflected portion and the diffracted portion of the two light signals comprise:
emitting a first one of the two light signals from at least one of the light sources at an initial wavelength range;
receiving a first signal from the optical sensor indicative of the detected light signal while the first one of the light signals at the initial wavelength range is being emitted;
emitting a second one of the two light signals from at least one of the light sources at a subsequent wavelength range, and
receiving a second signal from the optical sensor indicative of the detected light signal while the second one of the light signals at the subsequent wavelength range is being emitted.
